(19) United States
(12) Patent Application Publication
(10) Pub. No.: US 2009/0221604 A1
(43) Pub. Date: Sep. 3, 2009

(54) Thiazole Compounds and Their Use as PGD2 Antagonists

(76) Inventors: Neil Victor Harris, Essex (GB); George Hynd, Essex (GB); Sophie Gardan, Essex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/065,756

(22) PCT Filed: Sep. 8, 2006
(86) PCT No.: PCT/GB2006/003317

§ 371 Date: Sep. 11, 2008

(30) Foreign Application Priority Data
Sep. 9, 2005 (GB) 0518494.0

(51) Int. Cl. A61K 31/496; C07D 417/04; A61P 35/00; A61P 17/00; A61P 11/06

(52) U.S. Cl. 514/254.02; 544/369

(57) ABSTRACT

A compound for use as a PGD2 antagonist is of structural formula [1] in which: A represents a fully saturated or partially unsaturated monocyclic 5-7 membered ring containing one or two nitrogen atoms; B represents a direct bond, an optionally substituted methylene group, an optionally substituted nitrogen atom, oxygen, or S(O)ₙ, where n=0, 1, or 2; L represents a direct bond, or an optionally substituted alkylene or alkenylene group; R¹ represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group; R² represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group; X represents a carboxylic acid, tetrazole, 3-hydroxyisoxazole, hydroxamic acid, phosphinate, phosphonate, phosphonamide, sulfonic acid or a group of formula C(═O)NHSO₂Y or SO₂NHC(═O)Y; and Y represents an optionally substituted aryl or heteroaryl group or an optionally substituted alkyl or cycloalkyl group; or a N-oxide, pharmaceutically acceptable salt, solvate or prodrug thereof.

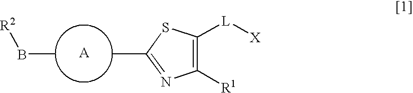

### FIELD OF THE INVENTION

This invention relates to thiazole compounds and their use in therapy.

### BACKGROUND OF THE INVENTION

Mast cells are known to play an important role in allergic and immune responses through the release of a number of mediators, such as histamine, leukotrienes, cytokines, prostaglandin D₂, etc (Boyce; Allergy Asthma Proc., 2004, 25, 27-30). Prostaglandin D₂ (PGD₂) is the major cyclooxygenase metabolite of arachadonic acid produced by mast cells in response to allergen challenge (Lewis et al; J. Immunol., 1982, 129, 1627-1631). It has been shown that PGD₂ production is increased in patients with systemic mastocytosis (Roberts; N. Engl. J. Med., 1980, 303, 1400-1404), allergic rhinitis (Naclerio et al; Am. Rev. Respir. Dis., 1983, 128, 597-602; Brown et al; Arch. Otolarynol. Head Neck Surg., 1987, 113, 179-183; Lebel et al; J. Allergy Clin. Immunol., 1988, 82, 869-877), bronchial asthma (Murray et al; N. Engl. J. Med., 1986, 315, 800-804; Liu et al; Am. Rev. Respir. Dis., 1990, 142, 126-132; Wenzel et al; J. Allergy Clin. Immunol., 1991, 87, 540-548), and urticaria (Heavey et al; J. Allergy Clin. Immunol., 1986, 78, 458-461). PGD₂ mediates it effects through two receptors, the PGD₂ (or DP) receptor (Boie et al; J. Biol. Chem., 1995, 270, 18910-18916) and the chemoattractant receptor-homologous molecule expressed on Th2 (or CRTH2) (Nagata et al; J. Immunol., 1999, 162, 1278-1289; Powell; Prostaglandins Luekot. Essent. Fatty Acids, 2003, 69, 179-185). Therefore, it has been postulated that agents that antagonise the effects of PGD₂ at its receptors may have beneficial effects in a number of disease states.

Significantly, it has been shown that transgenic mice that lack the PGD2 receptor produce lower concentrations of Th2 cytokines and reduced accumulation of eosinophils and lymphocytes in the bronchial alveolar lavage fluid compared to wild-type mice after antigen challenge (Matsuoka et al; Science, 2000, 287, 2013-2017). Furthermore, the PGD₂ receptor-deficient mice exhibited much reduced airway sensitivity to acetylcholine after antigen challenge when compared to wild type mice. In another experiment transgenic mice that overexpress in the lung the enzyme responsible for the synthesis of PGD₂ (PGD synthase) showed enhanced levels of Th2 cytokines and chemokines (IL-4, IL-5, and eotaxin) and an increased accumulation of lymphocytes and eosinophils in the bronchial alveolar lavage fluid when compared to wild-type mice (Fujitani et al; J. Immunol., 2002, 168, 443-449). It has also been shown that the PGD₂ receptor antagonist molecule S-5751 (Mitsumori et al; J. Med. Chem., 2003, 46, 2436-2445) inhibited both early (as assessed by sneezing, mucosal plasma exudation, and nasal blockage) and late (as assessed by eosinophil infiltration) phase nasal responses in an asthma model in the guinea pig after oral dosing (Arimura et al; J. Pharmacol. Exp. Ther., 2001, 298, 411-419). In addition, S-5751 alleviated allergen-induced plasma exudation in the conjunctiva in an allergic conjunctivitis model and antigen-induced eosinophil infiltration into the lung in an asthma model in the guinea pig. Finally, genetic variants with impaired expression of prostaglandin D receptor gene are linked to reduced asthma risk (Lilly et al; Am. J. Respir. Cell Mol. Biol., 2005, 33, 224-226). Therefore, it is expected that antagonists of the PGD₂ receptor may have utility in the treatment of a number of diseases.

A number of compounds have been reported to antagonise the effects of PGD₂ at the PGD₂ receptor. For example, S-5751, described above, and related compounds based around bicycloheptanyl and oxabicycloheptanyl cores have been described as PGD₂ receptor antagonists, and, in some cases, as mixed PGD₂ receptor and thromboxane (TXA₂ or TP) receptor antagonists (US 2004/0162323; WO2002/036583; WO2001/094309; WO2000/053573; WO1999/15502; WO1998/25915; WO1998/25919; WO1997/00853). Other compounds that have been described as PGD₂ receptor antagonists include: prostaglandin-like compounds (WO2004/074240), indole analogues (WO2004/078719; WO2003/022813; WO2003/022814; WO2001/066520) and fused indole ring systems bearing acidic functionalities (WO2005/056527; WO2004/111047; WO2004/039807; WO2003/062200; WO2002/094830; WO2002/008186; WO2001/079169; WO2004/039807), phenylacetic acid analogues WO2005/028455; WO2003/078409) and compounds based around a hydantoin core (EP284202). Surprisingly, we have now discovered that thiazole compounds of general structure [1] represent a novel class of PGD₂ receptor antagonists.

### SUMMARY OF THE INVENTION

One aspect of the invention is thiazole derivatives of general formula [1]:

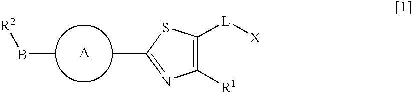

in which:

A represents a fully saturated or partially unsaturated monocyclic 5-7 membered ring containing one or two nitrogen atoms;

B represents a direct bond, an optionally substituted methylene group, an optionally substituted nitrogen atom, oxygen, or S(O)ₙ, where n=0, 1, or 2;

L represents a direct bond, or an optionally substituted alkylene or alkenylene group;

R¹ represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;

R² represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;

X represents a carboxylic acid, tetrazole, 3-hydroxyisoxazole, hydroxamic acid, phosphinate, phosphonate, phosphonamide, sulfonic acid or a group of formula C(═O)NHSO₂Y or SO₂NHC(═O)Y;

Y represents an optionally substituted aryl or heteroaryl group or an optionally substituted alkyl or cycloalkyl group;

and corresponding N-oxides, pharmaceutically acceptable salts, solvates and prodrugs of such compounds.

A second aspect of the invention is a pharmaceutical composition comprising a compound of formula [1] or an N-oxide, pharmaceutically acceptable salt, solvate or prodrug thereof, in admixture with a pharmaceutically acceptable carrier or excipient.

A third aspect of the invention is a compound of formula [1] or an N-oxide, pharmaceutically acceptable salt, solvate or prodrug thereof for use in therapy.

A fourth aspect of the invention is the use of a compound of formula [1], or an N-oxide, pharmaceutically acceptable salt, solvate or prodrug thereof, in the manufacture of a medicament for the treatment of a disease in which a PGD₂ antagonist can prevent, inhibit or ameliorate the pathology and/or symptomatology of the disease.

A fifth aspect of the invention is a method for treating a disease in a patient in which a PGD₂ antagonist can prevent, inhibit or ameliorate the pathology and/or symptomatology of the disease, which method comprises administering to the patient a therapeutically effective amount of compound of formula [1] or an N-oxide, pharmaceutically acceptable salt, solvate or prodrug thereof.

A sixth aspect of the invention is a method of preparing a compound of formula [1] or an N-oxide, pharmaceutically acceptable salt, solvate or prodrug thereof.

A seventh aspect of the invention is a method of making a pharmaceutical composition comprising combining a compound of formula [1], or an N-oxide, pharmaceutically acceptable salt, solvate or prodrug thereof, with a pharmaceutically acceptable carrier or excipient.

For purposes of the present invention, the following definitions as used throughout the description of the invention shall be understood to have the following meanings:

“Compounds of the invention”, and equivalent expressions, are meant to embrace compounds of general formula [1] as hereinbefore described, their N-oxides, their prodrugs, their pharmaceutically acceptable salts and their solvates, where the context so permits.

“Patient” includes both human and other mammals.

### DESCRIPTION OF THE INVENTION

For purposes of the present invention, the following chemical terms as used above, and throughout the description of the invention, and unless otherwise indicated, shall be understood to have the following meanings:

“Acyl” means a —CO-alkyl group in which the alkyl group is as described herein. Exemplary acyl groups include —COCH₃ and —COCH(CH₃)₂.

“Acylamino” means a —NR-acyl group in which R and acyl are as described herein. Exemplary acylamino groups include —NHCOCH₃ and —N(CH₃)COCH₃.

“Alkoxy” and “alkyloxy” means an —O-alkyl group in which alkyl is as defined below. Exemplary alkoxy groups include methoxy (OCH₃) and ethoxy (OC₂H₅).

“Alkoxycarbonyl” means a —COO-alkyl group in which alkyl is as defined below. Exemplary alkoxycarbonyl groups include methoxycarbonyl and ethoxycarbonyl.

“Alkyl” as a group or part of a group refers to a straight or branched chain saturated hydrocarbon group having from 1 to 12, preferably 1 to 6, carbon atoms, in the chain. Exemplary alkyl groups include methyl, ethyl, 1-propyl and 2-propyl.

“Alkenyl” as a group or part of a group refers to a straight or branched chain hydrocarbon group having from 1 to 12, preferably 1 to 6, carbon atoms and one carbon-carbon double bond in the chain. Exemplary alkenyl groups include ethenyl, 1-propenyl, and 2-propenyl.

“Alkylamino” means a —NH-alkyl group in which alkyl is as defined above. Exemplary alkylamino groups include methylamino and ethylamino.

“Alkylene” means an -alkyl-group in which alkyl is as defined previously. Exemplary alkylene groups include —CH₂—, —(CH₂)₂— and —C(CH₃)HCH₂—.

“Alkenylene” means an -alkenyl-group in which alkenyl is as defined previously. Exemplary alkenylene groups include —CH═CH—, —CH═CHCH₂—, and —CH₂CH═CH—.

“Alkylsufinyl” means a —SO-alkyl group in which alkyl is as defined above. Exemplary alkylsulfinyl groups include methylsulfinyl and ethylsulfinyl.

“Alkylsulfonyl” means a —SO₂-alkyl group in which alkyl is as defined above. Exemplary alkylsulfonyl groups include methylsulfonyl and ethylsulfonyl.

“Alkylthio” means a —S-alkyl group in which alkyl is as defined above. Exemplary alkylthio groups include methylthio and ethylthio.

“Aminoacyl” means a —CO—NR₂ group in which R is as herein described. Exemplary aminoacyl groups include —CONH₂ and —CONHCH₃.

“Aminoalkyl” means an alkyl-NH₂ group in which alkyl is as previously described. Exemplary aminoalkyl groups include —CH₂NH₂.

“Aminosulfonyl” means a —SO₂—NR₂ group in which R is as herein described. Exemplary aminosulfonyl groups include —SO₂NH₂ and —SO₂NHCH₃.

“Aryl” as a group or part of a group denotes an optionally substituted monocyclic or multicyclic aromatic carbocyclic moiety of from 6 to 14 carbon atoms, preferably from 6 to 10 carbon atoms, such as phenyl or naphthyl, and in one embodiment preferably phenyl. The aryl group may be substituted by one or more substituent groups.

“Arylalkyl” means an aryl-alkyl-group in which the aryl and alkyl moieties are as previously described. Preferred arylalkyl groups contain a C₁₋₄ alkyl moiety. Exemplary arylalkyl groups include benzyl, phenethyl and naphthlenemethyl.

“Arylalkyloxy” means an aryl-alkyloxy-group in which the aryl and alkyloxy moieties are as previously described. Preferred arylalkyloxy groups contain a C₁₋₄ alkyl moiety. Exemplary arylalkyl groups include benzyloxy.

“Aryl-fused-cycloalkyl” means a monocyclic aryl ring, such as phenyl, fused to a cycloalkyl group, in which the aryl and cycloalkyl are as described herein. Exemplary aryl-fused-cycloalkyl groups include tetrahydronaphthyl and indanyl. The aryl and cycloalkyl rings may each be substituted by one or more substituent groups. The aryl-fused-cycloalkyl group may be attached to the remainder of the compound of formula [1] by any available carbon atom.

“Aryl-fused-heterocycloalkyl” means a monocyclic aryl ring, such as phenyl, fused to a heterocycloalkyl group, in which the aryl and heterocycloalkyl are as described herein. Exemplary aryl-fused-heterocycloalkyl groups include tetrahydroquinolinyl, indolinyl, benzodioxinyl, benxodioxolyl, dihydrobenzofuranyl and isoindolonyl. The aryl and heterocycloalkyl rings may each be substituted by one or more substituent groups. The aryl-fused-heterocycloalkyl group may be attached to the remainder of the compound of formula [1] by any available carbon or nitrogen atom.

“Aryloxy” means an —O-aryl group in which aryl is described above. Exemplary aryloxy groups include phenoxy.

“Cyclic amine” means an optionally substituted 3 to 8 membered monocyclic cycloalkyl ring system where one of the ring carbon atoms is replaced by nitrogen, and which may optionally contain an additional heteroatom selected from O, S or NR (where R is as described herein). Exemplary cyclic amines include pyrrolidine, piperidine, morpholine, piperazine and N-methylpiperazine. The cyclic amine group may be substituted by one or more substituent groups.

“Cycloalkyl” means an optionally substituted saturated monocyclic or bicyclic ring system of from 3 to 12 carbon atoms, preferably from 3 to 8 carbon atoms, and more preferably from 3 to 6 carbon atoms. Exemplary monocyclic cycloalkyl rings include cyclopropyl, cyclopentyl, cyclohexyl and cycloheptyl. The cycloalkyl group may be substituted by one or more substituent groups.

“Cycloalkylalkyl” means a cycloalkyl-alkyl-group in which the cycloalkyl and alkyl moieties are as previously described. Exemplary monocyclic cycloalkylalkyl groups include cyclopropylmethyl, cyclopentylmethyl, cyclohexylmethyl and cycloheptylmethyl.

“Dialkylamino” means a —N(alkyl)₂ group in which alkyl is as defined above. Exemplary dialkylamino groups include dimethylamino and diethylamino.

“Halo” or “halogen” means fluoro, chloro, bromo, or iodo.

“Haloalkoxy” means an —O-alkyl group in which the alkyl is substituted by one or more halogen atoms. Exemplary haloalkyl groups include trifluoromethoxy and difluoromethoxy.

“Haloalkyl” means an alkyl group which is substituted by one or more halo atoms. Exemplary haloalkyl groups include trifluoromethyl.

“Heteroaryl” as a group or part of a group denotes an optionally substituted aromatic monocyclic or multicyclic organic moiety of from 5 to 14 ring atoms, preferably from 5 to 10 ring atoms, in which one or more of the ring atoms is/are element(s) other than carbon, for example nitrogen, oxygen or sulfur. Examples of such groups include benzimidazolyl, benzoxazolyl, benzothiazolyl, benzofuranyl, benzothienyl, furyl, imidazolyl, indolyl, indolizinyl, isoxazolyl, isoquinolinyl, isothiazolyl, oxazolyl, oxadiazolyl, pyrazinyl, pyridazinyl, pyrazolyl, pyridyl, pyrimidinyl, pyrrolyl, quinazolinyl, quinolinyl, tetrazolyl, 1,3,4-thiadiazolyl, thiazolyl, thienyl and triazolyl groups. The heteroaryl group may be substituted by one or more substituent groups. The heteroaryl group may be attached to the remainder of the compound of formula [1] by any available carbon or nitrogen atom.

“Heteroarylalkyl” means a heteroaryl-alkyl-group in which the heteroaryl and alkyl moieties are as previously described. Preferred heteroarylalkyl groups contain a lower alkyl moiety. Exemplary heteroarylalkyl groups include pyridylmethyl.

“Heteroarylalkyloxy” means a heteroaryl-alkyloxy-group in which the heteroaryl and alkyloxy moieties are as previously described. Preferred heteroarylalkyloxy groups contain a lower alkyl moiety. Exemplary heteroarylalkyloxy groups include pyridylmethyloxy.

“Heteroaryloxy” means a heteroaryloxy-group in which the heteroaryl is as previously described. Exemplary heteroaryloxy groups include pyridyloxy.

“Heteroaryl-fused-cycloalkyl” means a monocyclic heteroaryl group, such as pyridyl or furanyl, fused to a cycloalkyl group, in which heteroaryl and cycloalkyl are as previously described. Exemplary heteroaryl-fused-cycloalkyl groups include tetrahydroquinolinyl and tetrahydrobenzofuranyl. The heteroaryl and cycloalkyl rings may each be substituted by one or more substituent groups. The heteroaryl-fused-cycloalkyl group may be attached to the remainder of the compound of formula [1] by any available carbon or nitrogen atom.

“Heteroaryl-fused-heterocycloalkyl” means a monocyclic heteroaryl group, such as pyridyl or furanyl, fused to a heterocycloalkyl group, in which heteroaryl and heterocycloalkyl are as previously described. Exemplary heteroaryl-fused-heterocycloalkyl groups include dihydrodioxinopyridinyl, dihydropyrrolopyridinyl, dihydrofuranopyridinyl and dioxolopyridinyl. The heteroaryl and heterocycloalkyl rings may each be substituted by one or more substituent groups. The heteroaryl-fused-heterocycloalkyl group may be attached to the remainder of the compound of formula [1] by any available carbon or nitrogen atom.

“Heterocycloalkyl” means: (i) an optionally substituted cycloalkyl group of from 4 to 8 ring members which contains one or more heteroatoms selected from O, S or NR; (ii) a cycloalkyl group of from 4 to 8 ring members which contains CONR and CONRCO (examples of such groups include succinimidyl and 2-oxopyrrolidinyl). The heterocycloalkyl group may be substituted by one or more substituent groups. The heterocycloalkyl group may be attached to the remainder of the compound of formula [1] by any available carbon or nitrogen atom.

“Heterocycloalkylalkyl” means a heterocycloalkyl-alkyl-group in which the heterocycloalkyl and alkyl moieties are as previously described.

“Hydroxamate” means a group —C(O)NHOR where R is as described herein. Exemplary groups are —C(O)NHOH and —C(O)NHOCH₃.

“Lower alkyl” as a group means unless otherwise specified, an aliphatic hydrocarbon group which may be straight or branched having 1 to 4 carbon atoms in the chain, i.e. methyl, ethyl, propyl (n-propyl or iso-propyl) or butyl (n-butyl, isobutyl or tert-butyl).

“Phosphinate” means a —P(O)R(OR) group in which R is as described herein. Exemplary groups are —P(O)(OH)CH₃ and —P(O)(OH)H.

“Phosphonate” means a —P(O)(OH)OR group in which R is as described herein. Exemplary groups are —P(O)(OH)₂ and —P(O)(OH)OC₂H₅.

“Phosphonamide” means a —P(O)(OR)NR₂ group in which R is as described herein. An exemplary group is —P(O)(OH)NH₂.

“Sulfonate” means a —S(O)₂OR group where R is as described herein. Exemplary groups are —S(O)₂OH (sulfonic acid) and —S(O)₂OCH₃.

“Sulfonylamino” means a —NR-sulfonyl group in which R and sulfonyl are as described herein. Exemplary sulfonylamino groups include —NHSO₂CH₃.

“Pharmaceutically acceptable salt” means a physiologically or toxicologically tolerable salt and include, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that may be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that may be formed include hydrochlorides, hydrobromides, phosphates, acetates, citrates, lactates, tartrates, malonates, methanesulphonates and the like.

It will be understood that, as used in herein, references to the compounds of formula [1] are meant to also include the pharmaceutically acceptable salts.

“Prodrug” means a compound which is convertible in vivo by metabolic means (e.g. by hydrolysis, reduction or oxidation) to a compound of formula [1]. For example an ester prodrug of a compound of formula [1] containing a hydroxy group may be convertible by hydrolysis in vivo to the parent molecule. Suitable esters of compounds of formula [1] containing a hydroxy group, are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluenesulphonates, cyclohexylsulphamates and quinates. As another example an ester prodrug of a compound of formula [1] containing a carboxy group may be convertible by hydrolysis in vivo to the parent molecule. Examples of ester prodrugs are those described by F. J. Leinweber, Drug Metab. Res., 1987, 18, 379.

It will be understood that, as used in herein, references to the compounds of formula [1] are meant to also include the prodrug forms.

“Saturated” pertains to compounds and/or groups which do not have any carbon-carbon double bonds or carbon-carbon triple bonds.

The cyclic groups referred to above, namely, aryl, heteroaryl, cycloalkyl, aryl-fused-cycloalkyl, heteroaryl-fused-cycloalkyl, heterocycloalkyl, aryl-fused-heterocycloalkyl, heteroaryl-fused-heterocycloalkyl and cyclic amine may be substituted by one or more substituent groups. Suitable optional substituent groups include acyl (e.g. —COCH₃), alkoxy (e.g. —OCH₃), alkoxycarbonyl (e.g. —COOCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g —CF₃), alkyl (e.g. —CH₃ or —CH₂CH₃), —OH, —CHO, —NO₂, aryl (optionally substituted with alkoxy, haloalkoxy, halogen, alkyl or haloalkyl), heteroaryl (optionally substituted with alkoxy, haloalkoxy, halogen, alkyl or haloalkyl), heterocycloalkyl, aminoacyl (e.g. —CONH₂, —CONHCH₃), aminosulfonyl (e.g. —SO₂NH₂, —SO₂NHCH₃), acylamino (e.g. —NHCOCH₃), sulfonylamino (e.g. —NHSO₂CH₃), heteroarylalkyl, cyclic amine (e.g. morpholine), aryloxy, heteroaryloxy, arylalkyloxy (e.g. benzyloxy) and heteroarylalkyloxy.

Alkylene or alkenylene groups may be optionally substituted. Suitable optional substituent groups include alkoxy, alkylamino, alkylsulfinyl, alkylsulfonyl, alkylthio, —NH₂, aminoalkyl, arylalkyl, cyano, dialkylamino, halo, haloalkoxy, haloalkyl, alkyl, —OH, —CHO, and —NO₂.

Compounds of the invention may exist in one or more geometrical, optical, enantiomeric, diastereomeric and tautomeric forms, including but not limited to cis- and transforms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application or adaptation of known methods (e.g. asymmetric synthesis).

With reference to formula [1] above, particular and preferred embodiments are described below.

In one embodiment A is selected from the groups represented by formulae [2], [3], [4], [5], and [6]:

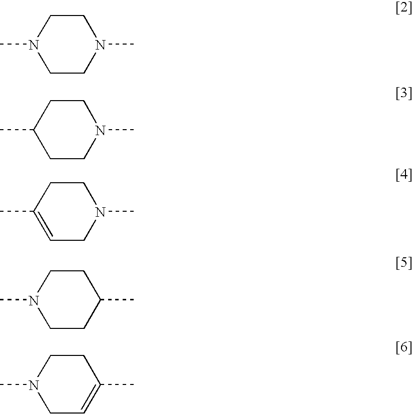

In a preferred embodiment A is a group of formula [2].

In another preferred embodiment A is a group of formula [3].

In one embodiment B is selected from a direct bond or an unsubstituted methylene group.

L may be a bond or may be selected from, for example, —CH₂—, —CH₂CH₂—, —CH₂CH₂CH₂—, —CH═CH—, —CH═CHCH₂—, and —CH₂CH═CH—. Presently a bond, —CH₂— and —CH₂CH₂— are preferred

In one embodiment R¹ is an optionally substituted monocyclic aryl or heteroaryl group.

In a preferred embodiment R¹ is an optionally substituted phenyl group.

In one embodiment R² is an optionally substituted monocyclic aryl or heteroaryl group.

In a preferred embodiment R² is an optionally substituted phenyl group.

Optional substituents in R¹ and R² may be selected from, for example, C₁-C₃ alkoxy such as methoxy and ethoxy, halo such as fluoro and chloro, cyano, C₁-C₃-alkyl such as methyl and ethyl, C₁-C₃-acylamino such as acetyl, and mono- or di-C₁-C₃-alkylamino such as mon- and dialkylamino wherein the alkyl parts are independently selected from methyl. and ethyl.

In a preferred embodiment X is a carboxylic acid group.

In another preferred embodiment X is a 5-tetrazolyl group.

In one embodiment compounds of the invention are:
1. 2-[4-(4-methoxyphenyl)piperazin-1-yl]-4-phenylthiazol-5-carboxylic acid;
2. (E)-3-{2-[4-(4-methoxyphenyl)piperazin-1-yl]-4-phenylthiazol-5-yl}acrylic acid;
3. 3-{2-[4-(4-methoxyphenyl)piperazin-1-yl]-4-phenylthiazol-5-yl}propionic acid;
4. [4-phenyl-2-(4-phenylpiperazin-1-yl)thiazole-5-yl]acetic acid;
5. {2-[4-(2-methoxyphenyl)piperazine-1-yl]-4-phenylthiazol-5-yl}acetic acid;
6. [2-(4-benzylpiperazin-1-yl)-4-phenylthiazol-5-yl]acetic acid;
7. N-{2-[4-phenyl-2-(4-phenylpiperazin-1-yl)thiazol-5-yl]acetyl}methanesulphonamide;
8. N-(3-{2-[4-(4-methoxyphenyl)piperazin-1-yl]-4-phenylthiazol-5-yl}propionyl)-benzenesulphonamide;
9. N-(2-{2-[4-(4-methoxyphenyl)piperazine-1-yl]-4-phenylthiazol-5-yl}acetyl)-benzenesulphonamide;
10. N-{2-[4-phenyl-2-(4-phenylpiperazin-1-yl)thiazole-5-yl]acetyl}benzenesulphonamide;
11. 4-phenyl-2-(4-phenylpiperidin-1-yl)thiazole-5-carboxylic acid;
12. 2-[4-(2-methoxyphenyl)piperazine-1-yl]-4-phenylthiazole-5-carboxylic acid;
13. 1-(4-methoxyphenyl)-4-[4-phenyl-5-(1H-tetrazol-5-yl)thiazol-2-yl]piperazine.
14. {2-[4-(4-methoxyphenyl)piperazine-1-yl]-4-phenylthiazol-5-yl}acetic acid;

### Utilities of the Invention

Whilst the compounds of the present invention have been shown to antagonise the effects of prostaglandin D₂ at the PGD₂ receptor according to the tests described in the Biological Methods section of this document, the mechanism of action by which the compounds act is not a limiting embodiment of the present invention. For example, compounds of the present invention may also have beneficial effects at other prostanoid receptors, such as the CRTH2 receptor or the thromboxane A₂ receptor.

The therapeutic application of these compounds is pertinent to any disease that is known to be at least partially mediated by the activation of the PGD₂ receptor. Examples of such diseases include, but are not limited to, asthma, allergic rhinitis, allergic conjunctivitis, nasal obstruction, atopic dermatitis, systemic mastocytosis, Crohn's disease, and ulcerative colitis. Other diseases in which a PGD₂ receptor antagonist may be of benefit include sleep disorders and other proliferative diseases such as cancer.

The present invention is also concerned with treatment of these conditions, and the use of compounds of the present invention for manufacture of a medicament useful in treating these conditions.

### Combinations

Other compounds may be combined with compounds of this invention of formula [1] for the prevention and treatment of prostaglandin-mediated diseases. Thus the present invention is also concerned with pharmaceutical compositions for preventing and treating PGD₂-mediated diseases comprising a therapeutically effective amount of a compound of the invention of formula [1] and one or more other therapeutic agents. Suitable therapeutic agents for a combination therapy with compounds of formula [1] include, but are not limited to: (1) corticosteroids, such as fluticasone, ciclesonide or budesonide; (2) β2-adrenoreceptor agonists, such as salmeterol, indacaterol or formoterol; (3) leukotriene modulators, for example leukotriene antagonists such as montelukast, zafirulast or pranlukast or leukotriene biosynthesis inhibitors such as Zileuton or BAY-1005; (4) anticholinergic agents, for example muscarinic-3 (M3) receptor antagonists such as tiotropium bromide; (5) phosphodiesterase-IV (PDE-IV) inhibitors, such as roflumilast or cilomilast; (6) antihistamines, for example selective histamine-1 (H1) receptor antagonists, such as fexofenadine, citirizine, loratidine or astemizole; (7) antitussive agents, such as codeine or dextramorphan; (8) non-selective COX-1/COX-2 inhibitors, such as ibuprofen or ketoprofen; (9) COX-2 inhibitors, such as celecoxib and rofecoxib; (10) VLA-4 antagonists, such as those described in WO97/03094 and WO97/02289; (11) TACE inhibitors and TNF-α inhibitors, for example anti-TNF monoclonal antibodies, such as Remicade and CDP-870 and TNF receptor immunoglobulin molecules, such as Enbrel; (12) inhibitors of matrix metalloprotease, for example MMP12; (13) human neutrophil elastase inhibitors, such as those described in WO2005/026124, WO2003/053930 and WO06/082412; (14) A2a agonists such as those described in EP1052264 and EP1241176 (15) A2b antagonists such as those described in WO2002/42298; (16) modulators of chemokine receptor function, for example antagonists of CCR3 and CCR8; (17) compounds which modulate the action of other prostanoid receptors, for example a CRTH2 receptor antagonist or a thromboxane A₂ antagonist; and (18) agents that modulate Th2 function, such as PPAR agonists.

The weight ratio of the compound of the formula (1) to the second active ingredient may be varied and will depend upon the effective dose of each ingredient. Generally, an effective dose of each will be used.

### Pharmaceutical Formulations

The present invention is also concerned with pharmaceutical formulations comprising one of the compounds as an active ingredient.

The magnitude of prophylactic or therapeutic dose of a compound may be determined by any suitable method known to one skilled in the art. It will be understood, however, that the specific amount for any particular patient will depend upon a variety of factors, including the activity of the specific compound that is used, the age, body weight, diet, general health and sex of the patient, time of administration, the route of administration, the rate of excretion, the use of any other drugs, and the severity of the disease undergoing treatment.

In general, the daily dose range will lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for the intravenous administration is employed, a suitable dosage range is from about 0.001 mg to about 25 mg (preferably from 0.01 mg to about 1 mg) of a compound of formula [1] per kg of body weight per day.

In the cases where an oral composition is employed, a suitable dosage range is, for example, from about 0.01 mg to about 300 mg of a compound of formula [1] per day, preferably from about 0.1 mg to about 30 mg per day. For oral administration, the compositions are preferably provided in the form of tablets containing from 0.01 to 1,000 mg, preferably 0.01, 0.05, 0.1, 0.5, 1.0, 2.5, 5.0, 10.0, 15.0, 20.0, 25.0, 30.0, 40.0, 50.0 or 1000.0 milligrams of the active ingredient for the symptomatic adjustment of the dosage to the patient to be treated.

The compounds of the invention may be administered by inhalation at a dose range from 0.0005 mg to 10 mg (preferably 0.005 mg to about 0.5 mg) per kg of body weight per day.

Another aspect of the present invention provides pharmaceutical compositions which comprise a compound of the invention and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the invention, additional active ingredient(s), and pharmaceutically acceptable excipients.

The pharmaceutical compositions of the present invention comprise a compound of the invention as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

Compounds of the invention may be used in combination with other drugs that are used in the treatment, prevention, suppression or amelioration of the diseases or conditions for which present compounds are useful. Such other drugs may be administered, by a route and in an amount commonly used therefore, contemporaneously or sequentially with a compound of the invention. When a compound of the invention is used contemporaneously with one or more other drugs, a pharmaceutical composition containing such other drugs in addition to the compound of the invention is preferred. Accordingly, the pharmaceutical compositions of the present invention include those that also contain one or more other active ingredients, in addition to a compound of the invention.

Any suitable route of administration may be employed for providing a mammal, especially a human, with an effective dosage of a compound of the present invention. In therapeutic use, the active compound may be administered by any convenient, suitable or effective route. The compositions include those compositions suitable for routes of administration known to those skilled in the art, and include oral, intravenous, rectal, parenteral, topical, ocular, nasal, buccal and pulmonary. Dosage forms include tablets, troches, dispersions, suspensions, solutions, capsules, creams, ointments, aerosols, and the like. They may be conveniently presented in unit dosage form and prepared by any of the methods well-known in the art of pharmacy.

For delivery by inhalation, the active compound is preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

By way of example, a composition of the invention may be prepared as a suspension for delivery from a nebuliser or as an aerosol in a liquid propellant, for example for use in a pressurised metered dose inhaler (PMDI). Propellants suitable for use in a PMDI are known to the skilled person, and include CFC-12, HFA-134a, HFA-227, HCFC-22 (CCl₂F₂) and HFA-152 (CH₂F₂) and isobutane.

Microparticles for delivery by administration may be formulated with excipients that aid delivery and release, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For example, in a dry powder formulation, microparticles may be formulated with large carrier particles that aid flow from the DPI into the lung. Suitable carrier particles are known, and include lactose particles; they may have a mass median aerodynamic diameter of greater than 90 μm.

In the case of an aerosol-based formulation, a preferred composition is:

| | | |
|---|---|---|
| Compound of the invention | 24 | mg/canister |
| Lecithin, NF Liq. Conc. | 1.2 | mg/canister |
| Trichlorofluoromethane, NF | 4.025 | g/canister |
| Dichlorodifluoromethane, NF | 12.15 | g/canister. |

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321).

In practical use, the compounds of formula [1] can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of formula [1] may also be administered by controlled release means and/or delivery devices such as those described in U.S. Pat. Nos. 3,845,770, 3,916,899, 3,536,809, 3,598,123, 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or moulding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Moulded tablets may be made by moulding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

The following are examples of representative pharmaceutical dosage forms for the compounds of formula [1]:

Injectable Suspension (I.M.):

| | | |
|---|---|---|
| Compound of formula [1] | 10 | mg/mL |
| Methylcellulose | 5.0 | mg/mL |
| Tween 80 | 0.5 | mg/mL |
| Benzyl alcohol | 9.0 | mg/mL |
| Benzalkonium chloride | 1.0 | mg/mL |

Plus water for injection to a total volume of 1 mL

500 mg Tablet:

| | | |
|---|---|---|
| Compound of formula [1] | 25 | mg/tablet |
| Microcrystalline Cellulose | 415 | mg/mL |
| Povidone | 14.0 | mg/mL |
| Pregelatinized Starch | 43.5 | mg/mL |
| Magnesium Stearate | 2.5 | mg/mL |

600 mg Capsule:

| | | |
|---|---|---|
| Compound of formula [1] | 25 | mg/tablet |
| Lactose Powder | 573.5 | mg/tablet |
| Magnesium Stearate | 1.5 | mg/tablet |

### Methods of Synthesis

The present invention is also concerned with processes for preparing the compounds of this invention.

The compounds of formula [1] of the present invention can be prepared according to the procedures of the following schemes and examples, using appropriate materials, and are further exemplified by the following specific examples. Moreover, by utilizing the procedures described with the disclosure contained herein, one of ordinary skill in the art can readily prepare additional compounds of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention of formula [1] may be isolated in the form of their pharmaceutically acceptable salts, such as those described previously herein above. The free acid form corresponding to isolated salts can be generated by acidification with a suitable acid such as acetic acid and hydrochloric acid and extraction of the liberated free acid into an organic solvent followed by evaporation. The free acid form isolated in this manner can be further converted into another pharmaceutically acceptable salt by dissolution in an organic solvent followed by addition of the appropriate base and subsequent evaporation, precipitation, or crystallisation.

It may be necessary to protect reactive functional groups (e.g. hydroxy, amino, thio or carboxy) in intermediates used in the preparation of compounds of formula [1] to avoid their unwanted participation in a reaction leading to the formation of compounds of formula [1]. Conventional protecting groups, for example those described by T. W. Greene and P. G. M. Wuts in “Protective groups in organic chemistry” John Wiley and Sons, 1999, may be used.

Compounds of the invention of formula [1a] in which L represents a direct bond or an optionally substituted alkylene group may be conveniently prepared by the reaction of a thioamide compound of formula [7] with a compound of formula [8], in which R³ represents an appropriate ester protecting group, to give a compound of formula [9], followed by deprotection of the ester group R³ in compounds of formula [9] to give the desired carboxylic acid of formula [1a]. Suitable ester protecting groups include, for example, R³=methyl or ethyl, which may be removed by acid- or base-catalysed aqueous hydrolysis, R³=benzyl, which may be removed by catalytic hydrogenation, or R³=tert-butyl, which may be removed by treatment with a strong non-aqueous acid such as trifluoroacetic acid/dichloromethane mixtures, or a solution of hydrogen chloride in dioxane.

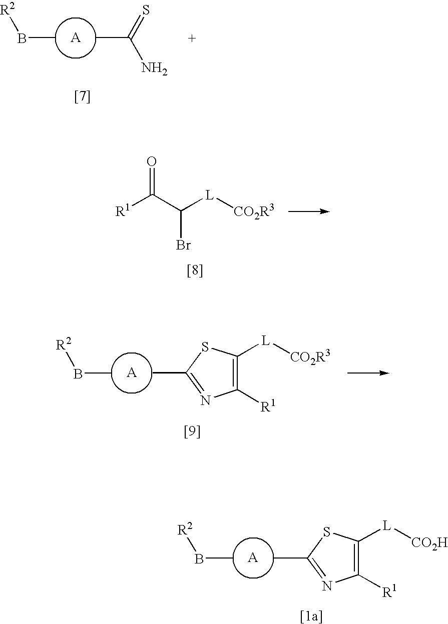

Intermediate compounds of formula [7] in which the thioamide group is attached to group A through a nitrogen atom, may be prepared, for example, by the reaction of a compound of formula [10] with an isothiocyanate of formula [11], in which R⁴ is a suitable protecting group. Suitable protecting groups at R⁴ include benzoyl and trimethylsilyl, and such protecting groups may be lost spontaneously during the reaction of [10] with [11], or may require a separate deprotection step such as aqueous hydrolysis for their removal.

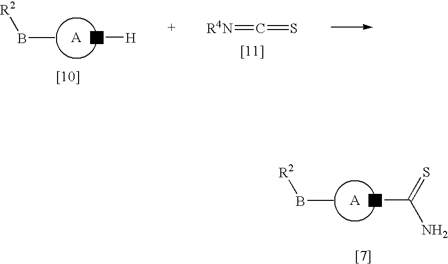

Intermediate compounds of formula [7] in which the thioamide group is attached to group A through a carbon atom, may be prepared, for example, by the reaction of a nitrile of formula [12] with hydrogen sulphide.

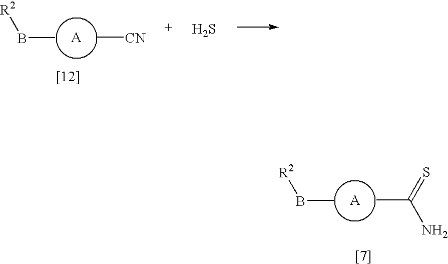

Intermediate compounds of formula [8] may be prepared by, for example, the reaction of ω-keto esters of formula [13] with an appropriate brominating agent, for example molecular bromine.

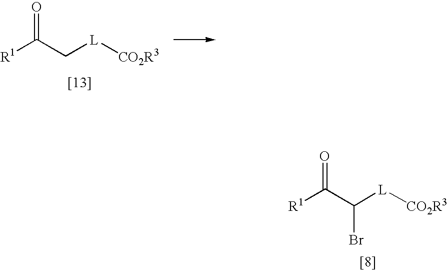

Compounds of the invention of formula [1b] in which group A is attached to the thiazole ring through a nitrogen atom, in which X represents a carboxylic acid group and in which L represents a direct bond or an optionally substituted alkylene group may be conveniently prepared by the reaction in an inert solvent, usually under elevated temperatures, of a cyclic amine of formula [10] with a thiazole analogue of formula [14] in which R⁵ is a leaving group; suitable leaving groups at R⁵ include chloro, bromo, alkylsulphinyl, and alkylsulphonyl.

Alternatively the reaction of intermediate [14], in which R⁵ is a halo group such as chloro or bromo, with an cyclic amine intermediate of formula [10] may be achieved in the presence of a palladium catalyst such as a mixture of palladium bis(trifluoroacetate) and tri(tert-butyl)phosphine.

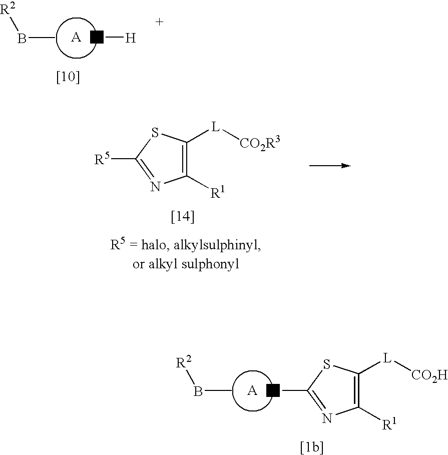

It will be understood by those who are practiced in the art that the transformation of intermediate [14] to compound [1b] by reaction with intermediate [10] may be performed either on the carboxylic acid form of [14] (i.e. R³=H in this scheme) or on a protected form of [14], such as an ester (i.e. R³=methyl or ethyl, for example), as may prove to be most convenient. It is to be understood that if the reaction is carried out on a protected form of intermediate [14] an appropriate deprotection step will be required to obtain the desired compound [1b] of the invention.

Intermediates of formula [14] in which R⁵ is a halogen atom such as chloro or bromo may be prepared from the reaction of an intermediate ω-keto ester of formula [8] with thiourea to give a 2-aminothiazole of formula [15], followed by diazotisation of the 2-amino group to give the required intermediate of formula [14].

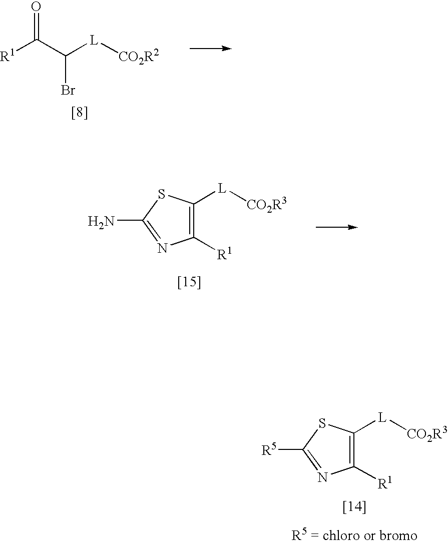

Alternatively, intermediates of formula [14] in which R⁵ is alkylsulphinyl or alkylsulphonyl may be prepared from intermediates of formula [14] in which R⁵ is chloro or bromo atom by reaction with a thiol of formula [16] to give an intermediate of formula [17], followed by oxidation with a suitable reagent such as hydrogen peroxide or 3-chloroperoxybenzoic acid to give the required intermediate [14].

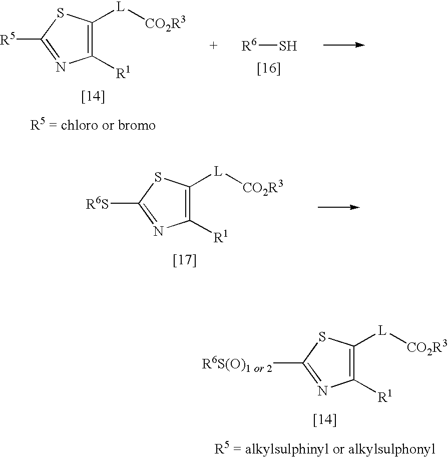

Compounds of the invention of formula [1c] in which group A is attached to the thiazole ring through an unsaturated carbon atom (for example A is a group of formula [6]), in which L represents a direct bond or an optionally substituted alkylene group may be conveniently prepared by the reaction between an intermediate of formula [14] in which R⁵ is a halo atom such as chloro or bromo, and a substituted alkene of formula [18], in which R⁷ is a suitable metal-containing group such as a boronate ester or a trialkyl- or triarylstanne, in the presence of a suitable palladium catalyst such as tris(dibenzylideneacetone)dipalladium.

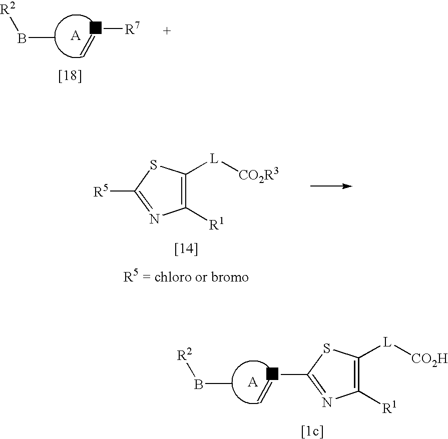

It will be understood by those who are practiced in the art that the transformation of intermediate [14] to compound [1c] by reaction with intermediate [18] may be performed either on the carboxylic acid form of [18] (i.e. R³=H in this scheme) or on a protected form of [18], such as an ester (i.e. R³=methyl or ethyl, for example), as may prove to be most convenient. It is to be understood that if the reaction is carried out on a protected form of intermediate [14] an appropriate deprotection step will be required to obtain the desired compound [1c] of the invention.

Compounds of the invention of formula [1d] in which group A is attached to the thiazole ring through a saturated carbon atom (for example A is a group of formula [5]), in which X represents a carboxylic acid group and in which L represents a direct bond or an optionally substituted alkylene group may be conveniently prepared by the reduction of compounds of formula [1c], by, for example, catalytic hydrogenation. It will be understood by those practiced in the art that the transformation of [1c] to [1d] may be conveniently performed either on the carboxylic acid form of [1c] or on a protected version of [1c], for example an ester. It is to be understood that if the reaction is carried out on a protected form of compound [1c] a deprotection step will be required to obtain compound [1d].

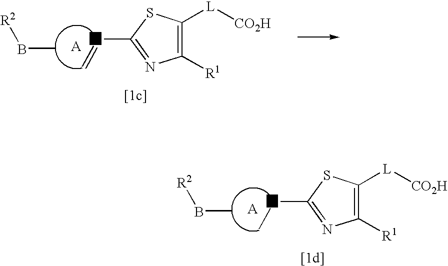

Compounds of the invention of formula [1e] in which X represents a carboxylic acid group, in which L represents an optionally substituted alkylene group, and in which group A is attached to the thiazole ring through a nitrogen atom (for example group A is represented by formulae [2], [3], or [4]) may be conveniently prepared by the reaction of a substituted ω-keto ester compound of formula [19], in which R³ represents an appropriate ester protecting group, with ammonia, followed by deprotection of the ester group R³ to give the desired carboxylic acid of formula [1e]. Suitable ester protecting groups include, for example, R³=methyl or ethyl, which may be removed by acid- or base-catalysed aqueous hydrolysis, R³=benzyl, which may be removed by catalytic hydrogenation, or R³=1,1-dimethylethyl, which may be removed by treatment with a strong non-aqueous acid such as trifluoroacetic acid/dichloromethane mixtures, or a solution of hydrogen chloride in dioxane.

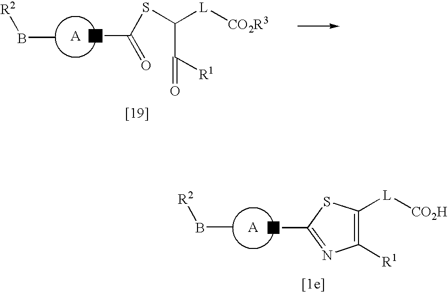

Intermediates of formula [19] may be prepared by, for example, the reaction of a carbothioic acid of formula [20] with an ω-keto ester compound of formula [8], in which R³ represents an appropriate ester protecting group.

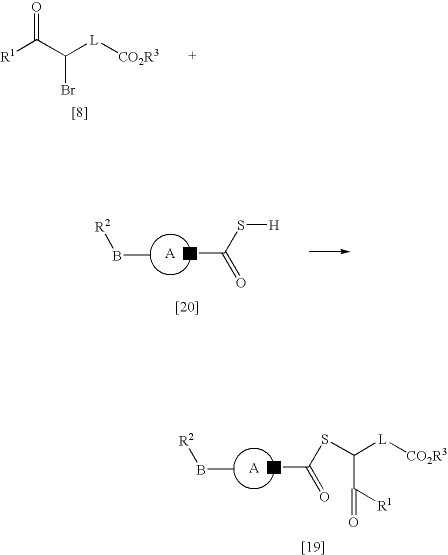

Compounds of the invention of formula [1f] in which X represents a carboxylic acid group and in which L represents an optionally substituted alkenylene group (for example an optionally substituted ethylene group) may be conveniently prepared by the reaction of an aldehyde or ketone of formula [21], in which R⁸ is a hydrogen or alkyl group, with, for example, an appropriate phosphorane or the anion of an appropriate phosphonate ester, followed by deprotection of the ester group at R³ in intermediate [22] to give the required compound of formula [1f]. Furthermore, it will be understood by those practiced in the art that compounds of the invention of formula [1g], in which X is a carboxylic acid group and L is an optionally substituted alkylene group comprising a chain of at least two carbon atoms, may be prepared by the reduction of compounds of the invention of formula [1f] by, for example, catalytic hydrogenation. It is to be understood that the transformation of [1f] to [1g] may be conveniently carried out either on the carboxylic acid form of [1f] or on a protected form of [1f], for example an ester. It is to be understood that if the reaction is carried out on a protected form of [1f] an appropriate deprotection step will be required to obtain the desired compound of the invention of formula [1g].

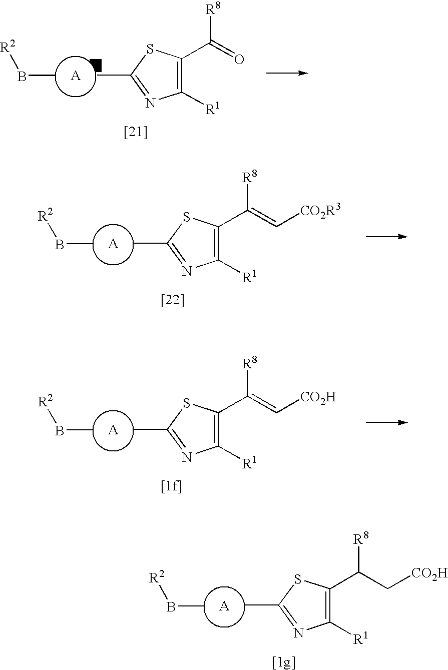

Intermediates of formula [21] in which R⁸=H may be prepared by, for example, the reduction of compounds of formula [23] in which R³ is a hydrogen or alkyl group, to an intermediate alcohol of formula [24], followed by oxidation of the intermediate [24] to the desired compound of formula [21].

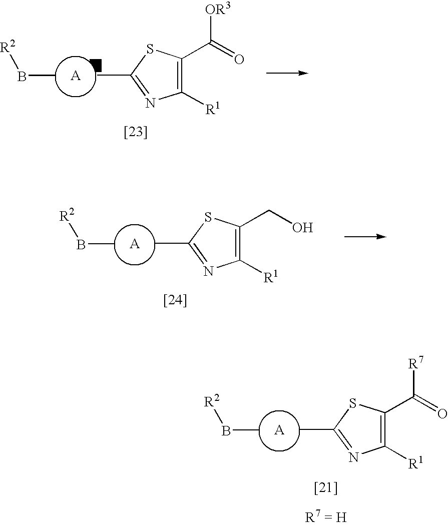

It will be understood by those practiced in the art that compounds of the invention may be prepared by transformations of other compounds of the invention. For example, compounds of the invention of formula [1h] may be prepared by the reaction between compounds of formula [1a] and an sulphonamide of formula Y—SO₂NH₂. This reaction may be conveniently performed in the presence of an appropriate condensing agent, for example 1-(3-dimethylaminopropyl)-3-ethylcarbodiimide hydrochloride.

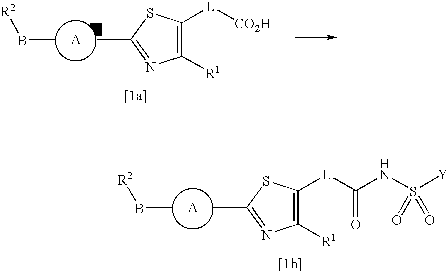

Compounds of the invention of formula [1i] in which the group A is attached to the group R²—B through a nitrogen atom (for example group A is a group of formula [2], [5], or [6]) may conveniently be prepared from an intermediate of formula [25]. For example, when B is a direct bond compounds of formula [1i] may be prepared by the reaction between a compound of formula [25] and an appropriate compound of formula R²-halogen. It may be convenient to perform this reaction in the presence of a suitable palladium catalyst, or it may be convenient to carry out this reaction under thermal conditions, depending on the exact nature of the compound R²-halogen.

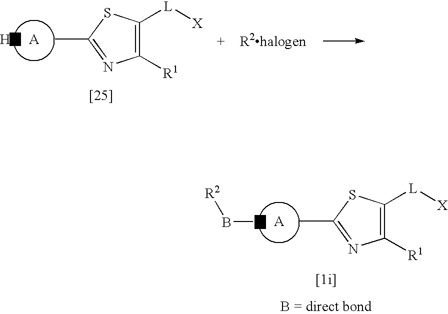

Similarly, when group B in formula [1i] represents an unsubstituted methylene group, compounds of formula [1i] may be conveniently prepared by, for example, the reaction between a compound of formula [25] and an aldehyde of formula R²—CHO in the presence of a suitable reducing agent such as sodium cyanoborohydride.

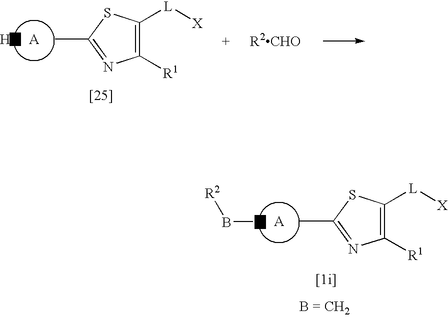

Intermediate compounds of formula [25] may be conveniently prepared by the preparation of compounds of formula [1i] in which the group R²—B represents a suitable protecting group, followed by a deprotection step to yield the required intermediate [25]. Suitable protecting groups at R²—B may include, for example, benzyl, which may be removed by catalytic hydrogenation, or tert-butyloxycarbonyl, which may be removed by treatment with a strong non-aqueous acid such as trifluoroacetic acid/dichloromethane mixtures, or a solution of hydrogen chloride in dioxane.

### Biological Methods

Compounds of the invention of formula [1] can be tested using the following biological test methods to determine their ability to displace PGD₂ from the PGD₂ receptor and for their ability to antagonise the functional effects of PGD₂ at the PGD₂ receptor in a whole cell system.

### Radioligand Binding Assay Using LS174T Membranes

The receptor binding assay is performed in a final volume of 200 μl binding buffer (10 mM BES (pH7.4), 1 mM EDTA, 10 mM manganese chloride, 0.01% BSA) using 1 nM [³H]-PGD₂ (Amersham Biosciences UK Ltd) as the radioligand. Ligands are added in assay buffer containing a constant volume of DMSO (1% by volume). Total binding is determined using 1% by volume of DMSO in assay buffer and non-specific binding is determined using 10 μM of unlabeled PGD₂ (sigma). The reaction is initiated with 100 μg LS174T cell membranes and the mixture incubated for 90 minutes at room temperature. The reaction is terminated by rapid filtration through GF/C filters prewetted with brij 35 (1% by volume) using a Packard Cell harvester and the filter washed with 600 μl/well of wash buffer (10 mM BES pH7.4 and 120 mM NaCl). The residual radioligand bound to the filter is determined using a Topcount liquid scintillation counter (Perkin Elmer). Compound IC₅₀ value was determined using a 6-point dose response curve in duplicate with a semi-log compound dilution series. IC₅₀ calculations were performed using Excel and XL fit (Microsoft) and this value is used to determine a Kᵢ value for the test compound using the Cheng-Prusoff equation. Compounds of the invention of formula [1] typically show Kᵢ values of less than 10 μM in this system.

### Functional Assay in LS174T Cells

LS174T cells are grown to confluence on the day of the assay. The cells are washed with PBS, incubated for 5 minutes in cell dissociation buffer, harvested by centrifugation at 300 g for 5 minutes at room temperature and resuspended at 2×10⁶/ml in stimulation buffer (Hanks balanced salt solution containing 5 mM HEPES, 0.1% BSA, 0.2 mM phosphodiesterase inhibitor (IBMX) adjusted to pH 7.4). The assay was performed in a final incubation volume of 25 μl in 384 well opaque optiplates (Perkin Elmer) using the ALPHAScreen (amplified luminescent proximity homogenous assay) cAMP assay kit (Perkin Elmer). To detect antagonists and determine compound IC₅₀, cells are incubated for 30 minutes with increasing concentrations of compound at room temperature in the presence of the agonist at its EC₈₀ (2.5 nM PGD₂). Compounds are added in stimulation buffer containing a constant volume of DMSO (0.4% by volume). The reaction is stopped by addition of lysis buffer (distilled water containing 5 mM HEPES, 0.1% BSA and 0.3% Tween-20, adjusted to pH 7.4) and the amount of cAMP measured using the Fusion-α (Perkin Elmer). Compound IC₅₀ was determined using an 8-point dose response curve in triplicate with a semi-log compound dilution series. Maximal stimulation and inhibition of cAMP production was determined in the presence of 2.5 nM PGD₂ and 10 μM S-5175 (Shionogi) respectively and all compound responses are determined as a percentage of this. IC₆₀ calculations were performed using Excel and XL fit (Microsoft) and this value is used to determine a Ki value for the test compound using the Cheng-Prusoff equation. Compounds of the invention of formula [1] typically show Kᵢ values of less than 10 μM in this system.

### EXAMPLES

The invention will now be described in detail with reference to the following examples. It will be appreciated that the invention is described by way of example only and modification of detail may be made without departing from the scope of the invention.

¹H NMR spectra were recorded at ambient temperature using Varian Mercury 200 (200 MHz) spectrometer (Method A) or a Varian Unity Inova (400 MHz) spectrometer a with a triple resonance 5 mm probe spectrometer (Method B). Chemical shifts are expressed in ppm relative to tetramethylsilane. The following abbreviations have been used: br=broad signal, s=singlet, d=doublet, dd=double doublet, t=triplet, q=quartet, m=multiplet.

Mass Spectrometry (LCMS) experiments to determine retention times and associated mass ions were performed on a Micromass Platform LCT spectrometer with positive ion electrospray and single wavelength UV 254 nm detection using a Higgins Clipeus C18 5 □m 100×3.0 mm column and a 2 mL/minute flow rate. The initial solvent system was 95% water containing 0.1% formic acid (solvent A) and 5% acetonitrile containing 0.1% formic acid (solvent B) for the first minute followed by a gradient up to 5% solvent A and 95% solvent B over the next 14 minutes. The final solvent system was held constant for a further 2 minutes.

Microwave experiments were carried out using a Personal Chemistry Smith Synthesizer™, which uses a single-mode resonator and dynamic field tuning, both of which give reproducibility and control. Temperatures from 40-250° C. can be achieved, and pressures of up to 20 bar can be reached. Two types of vial are available for this processor, 0.5-2.0 mL and 2.0-5.0 mL.

Reverse-phase preparative HPLC purifications were carried out using Genesis 7 micron C-18 bonded silica stationary phase in columns 10 cm in length and 2 cm internal diameter. The mobile phase used was mixtures of acetonitrile and water (both buffered with 0.1% v/v trifluoroacetic acid) with a flow rate of 10 mL per minute and typical gradients of 40 to 90% organic modifier ramped up over 30 to 40 minutes. Fractions containing the required product (identified by LC-MS analysis) were pooled, the organic fraction removed by evaporation, and the remaining aqueous fraction lyophilised, to give the final product.

### Example 1

### 2-[4-(4-methoxyphenyl)piperazin-1-yl]-4-phenylthiazol-5-carboxylic acid

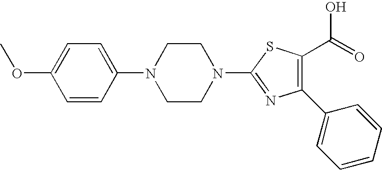

### Preparation (1a) 4-(4-methoxyphenyl)piperazinecarbothioic acid amide

4-(4-Methoxyphenyl)piperazinecarbothioic acid amide was prepared following a literature procedure (Nagarajan et al, Ind. J. Chem., 1969, 7, 1195-1197).

### Preparation (1b) ethyl 2-[4-(4-methoxyphenyl)piperazin-1-yl]-4-phenylthiazol-5-carboxylate

A mixture of 4-(4-methoxyphenyl)piperazinecarbothioic acid amide (1.5 g), ethyl 2-bromo-3-oxo-3-phenylpropionate (1.6 g) and ethanol (10 mL) was heated at reflux for five minutes. The ethanol was removed under reduced pressure and an aqueous solution of sodium hydrogen carbonate (5% w/v solution in water, 30 mL) was added to the residue. The resultant precipitate was collected by filtration to give ethyl 2-[4-(4-methoxyphenyl)piperazin-1-yl]-4-phenylthiazol-5-carboxylate as a white powder, 2.15 g.

¹H NMR (CDCl₃; Method A): δ 1.16 (t, J=7.2 Hz, 3H), 2.94-3.32 (m, 4H), 3.47-3.76 (m, 4H), 3.67 (s, 3H), 4.09 (q, J=7.2 Hz, 3H), 6.83 (d, J=8.2 Hz, 2H), 6.98 (d, J=8.2 Hz, 2H), 7.25-7.54 (m, 3H), 7.54-7.89 (m, 2H).

### Preparation (1c) 2-[4-(4-methoxyphenyl)piperazin-1-yl]-4-phenylthiazol-5-carboxylic acid

Ethyl 2-[4-(4-methoxyphenyl)piperazin-1-yl]-4-phenylthiazol-5-carboxylate (0.2 g) was treated with a solution of lithium hydroxide (0.5 g) in water (5.0 mL) and ethanol (5.0 mL), and the resulting mixture was stirred at 60° C. for three hours. Ethanol was removed under reduced pressure and the pH of the solution was adjusted to 6 by the addition of acetic acid. The precipitate was collected by filtration to give 2-[4-(4-methoxyphenyl)piperazin-1-yl]-4-phenylthiazol-5-carboxylic acid as a white powder, 0.15 g.

¹H NMR (DMSO-d6; Method B): δ 3.15 (m, 4H), 3.65 (m, 4H), 3.70 (s, 3H), 6.85 (d, J=9.1 Hz, 2H), 6.95 (d, J=9.1 Hz, 2H), 7.35 (m, 3H), 7.65-7.70 (m, 2H), 12.50 (br s, 1H).

MS: ESI (+ve): 396 (M+H)⁺, Retention time 10.8 min.

### Example 2

### (E)-3-{2-[4-(4-methoxyphenyl)piperazin-1-yl]-4-phenylthiazol-5-yl}acrylic acid

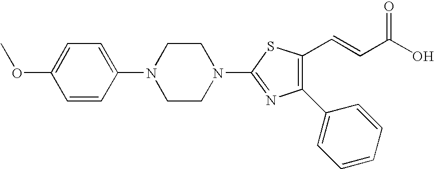

### Preparation (2a) {2-[4-(4-methoxyphenyl)piperazine-1-yl]-4-phenylthiazol-5-yl}methanol

Ethyl 2-[4-(4-methoxyphenyl)piperazin-1-yl]-4-phenylthiazol-5-carboxylate (compound from Preparation (1b), 0.80 g) was added to a suspension of lithium aluminium hydride (1.6 g) in tetrahydrofuran (15 mL) and the resulting mixture was stirred at room temperature for one hour. The mixture was diluted with water (5.0 mL) and extracted with ethyl acetate. The combined extracts were dried over sodium sulphate and the solvent removed under reduced pressure to give {2-[4-(4-methoxyphenyl)piperazine-1-yl]-4-phenylthiazol-5-yl}methanol as a white powder, 0.43 g.

¹H NMR (CDCl₃; Method A): δ 2.98-3.25 (m, 4H), 3.41-3.65 (m, 4H), 3.68 (s, 3H), 4.56 (d, J=5.2 Hz, 2H), 5.45 (t, J=5.2 Hz, 2H), 6.81 (d, J=8.1 Hz, 2H), 6.96 (d, J=8.1 Hz, 2H), 7.23-7.49 (m, 3H), 7.49-7.73 (m, 2H).

### Preparation (2b) 2-[4-(4-methoxyphenyl)piperazine-1-yl]-4-phenylthiazol-5-carbaldehyde

A mixture of {2-[4-(4-methoxyphenyl)piperazine-1-yl]-4-phenylthiazol-5-yl}methanol (0.40 g), manganese dioxide (4.0 g) and benzene (15 mL) was stirred at room temperature for 30 minutes. The mixture was filtered and solvent removed under reduced pressure give 2-[4-(4-methoxyphenyl)piperazine-1-yl]-4-phenylthiazol-5-carbaldehyde as a colourless oil, 0.36 g.

¹H NMR (CDCl₃; Method A): δ 2.98-3.36 (m, 4H), 3.63-3.83 (m, 4H), 3.67 (s, 3H), 6.83 (d, J=8.1 Hz, 2H), 6.96 (d, J=8.1 Hz, 2H), 7.41-7.65 (m, 3H), 7.65-7.89 (m, 2H), 9.65 (s, 1H).

### Preparation (2c) methyl (E)-3-{2-[4-(4-methoxyphenyl)piperazin-1-yl]-4-phenylthiazol-5-yl}acrylate

Trimethylphosphonoacetate (0.20 mL) was added to a solution of potassium tert-butoxide (0.13 g) in dimethylsulfoxide (5.0 mL) and the resulting mixture was stirred at room temperature for ten minutes. 2-[4-(4-Methoxyphenyl)piperazine-1-yl]-4-phenylthiazol-5-carbaldehyde (0.33 g) was then added and the mixture stirred at room temperature for one hour. The reaction mixture was diluted with water (50 mL) and the resulting precipitate was collected by filtration to give methyl (E)-3-{2-[4-(4-methoxyphenyl)piperazin-1-yl]-4-phenylthiazol-5-yl}acrylate as a white powder, 0.25 g.

¹H NMR (CDCl₃; Method A): δ 2.96-3.27 (m, 4H), 3.49-3.83 (m, 4H), 3.61 (s, 3H), 3.65 (s, 3H), 5.81 (d, J=15.6 Hz, 1H), 6.85 (d, J=8.1 Hz, 2H), 6.96 (d, J=8.1 Hz, 2H), 7.45-7.63 (m, 5H), 7.61 (d, J=15.2 Hz, 1H).

### Preparation (2d) (E)-3-{2-[4-(4-methoxyphenyl)piperazin-1-yl]-4-phenylthiazol-5-yl}acrylic acid

Methyl (E)-3-{2-[4-(4-methoxyphenyl)piperazin-1-yl]-4-phenylthiazol-5-yl}acrylic acid methyl ester (0.2 g) was treated with a solution of lithium hydroxide (0.5 g) in water (5.0 mL) and ethanol (5.0 mL), and the resulting mixture was stirred at 60° C. for three hours. The ethanol was removed under reduced pressure and pH of the solution was adjusted to 6 by the addition of acetic acid. The precipitate was collected by filtration to give (E)-3-{2-[4-(4-methoxyphenyl)piperazin-1-yl]-4-phenylthiazol-5-yl}acrylic acid as a yellow powder, 0.15 g.

¹H NMR (DMSO-d6; Method B): δ 3.10 (m, 4H), 3.65 (m, 7H), 5.70 (d, J=15.2 Hz, 1H), 6.80 (d, J=9.0 Hz, 2H), 6.90 (d, J=9.0 Hz, 2H), 7.20-7.55 (m, 6H), 12.20 (br s, 1H).

MS: ESI (+ve): 422 (M+H)⁺, Retention time 11.6 min.

### Example 3

### 3-{2-[4-(4-methoxyphenyl)piperazin-1-yl]-4-phenylthiazol-5-yl}propionic acid

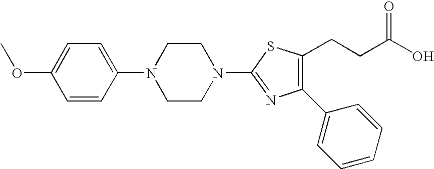

### Preparation (3a) methyl 3-{2-[4-(4-methoxyphenyl)piperazin-1-yl]-4-phenylthiazol-5-yl}propionate

A solution of methyl (E)-3-{2-[4-(4-methoxyphenyl)piperazin-1-yl]-4-phenylthiazol-5-yl}acrylate (compound of Preparation (2c), 0.23 g) in methanol (20 mL) was treated with palladium on carbon (10% w/w, 0.10 g) and the resulting mixture was shaken under an atmosphere of hydrogen (5 bar) for four days. The catalyst was removed by filtration and the filtrate was concentrated under reduced pressure to give methyl 3-{2-[4-(4-methoxyphenyl)piperazin-1-yl]-4-phenylthiazol-5-yl}propionate as a white powder, 0.21 g.

¹H NMR (CDCl₃; Method A): δ 2.63 (t, J=7.0 Hz, 2H), 2.94-3.29 (m, 6H), 3.29-3.67 (m, 4H), 3.58 (s, 3H), 3.68 (s, 3H), 6.85 (d, J=8.2 Hz, 2H), 7.03 (d, J=8.2 Hz, 2H), 7.21-7.78 (m, 5H).

### Preparation (3b) 3-{2-[4-(4-methoxyphenyl)piperazin-1-yl]-4-phenylthiazol-5-yl}propionic acid

Methyl 3-{2-[4-(4-methoxyphenyl)piperazin-1-yl]-4-phenylthiazol-5-yl}propionate (0.2 g) was treated with a solution of lithium hydroxide (0.5 g) in water (5.0 mL) and ethanol (5.0 mL), and the resulting mixture was stirred at 60° C. for three hours. The ethanol was removed under reduced pressure and pH of the solution was adjusted to 6 by the addition of acetic acid. The precipitate was collected by filtration to give 3-{2-[4-(4-methoxyphenyl)piperazin-1-yl]-4-phenylthiazol-5-yl}propionic acid as a white powder, 0.15 g.

¹H NMR (DMSO-d6; Method B): δ 2.55 (t, J=7.4 Hz, 2H), 3.00 (t, J=7.4 Hz, 2H), 3.15 (m, 4H), 3.50 (m, 4H), 3.70 (s, 3H), 6.85 (d, J=9.1 Hz, 2H), 6.95 (d, J=9.1 Hz, 2H), 7.35 (m, 1H), 7.40-7.45 (m, 2H), 7.55 (m, 2H).

MS: ESI (+ve): 424 (M+H)⁺, Retention time 11.8 min.

### Example 4

### [4-phenyl-2-(4-phenylpiperazin-1-yl)thiazole-5-yl]acetic acid

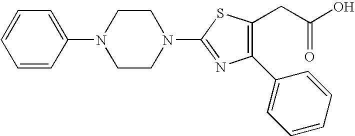

### Preparation (4a) 4-phenylpiperazin-1-carbothioic acid amide

4-phenylpiperazinecarbothioic acid amide was prepared following a literature procedure (Nagarajan et al, Ind. J. Chem., 1969, 7, 1195-1197).

### Preparation (4b) [4-phenyl-2-(4-phenylpiperazin-1-yl)thiazole-5-yl]acetic acid

A solution of 4-phenylpiperazin-1-carbothioic acid amide (0.20 g) and methyl 3-bromo-4-oxo-4-phenylbutanoate (0.25 g) in ethanol (6.0 mL) was heated at reflux for 45 minutes. The reaction mixture was concentrated under reduced pressure and the residue was treated with a solution of sodium hydroxide (0.1 g) in water (5.0 mL) and methanol (5.0 mL). This mixture was stirred at 35° C. for live hours and then the methanol was removed under reduced pressure. The pH of the residue was adjusted to 6 by the addition of dilute hydrochloric acid and the resulting precipitate was collected by filtration, washed with water and dried to give [4-phenyl-2-(4-phenylpiperazin-1-yl)thiazole-5-yl]acetic acid as a white powder, 0.24 g.

¹H NMR (DMSO-d6; Method B): δ 3.20 (m, 4H), 3.50 (m, 4H), 3.65 (s, 2H), 6.75 (m, 1H), 6.95 (m, 2H), 7.15-7.20 (m, 2H), 7.30 (m, 1H), 7.35-7.40 (m, 2H), 7.50 (m, 2H).

MS: ESI (+ve): 380 (M+H)⁺, Retention time 11.8 min.

### Example 5

### {2-[4-(2-methoxyphenyl)piperazine-1-yl]-4-phenylthiazol-5-yl}acetic acid

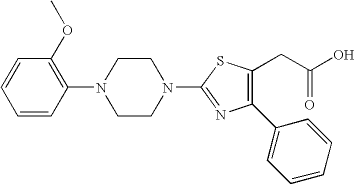

### Preparation (5a) 4-(2-methoxyphenyl)piperazinecarbothioic acid amide

4-(2-Methoxyphenyl)piperazinecarbothioic acid amide was prepared following a literature procedure (Nagarajan et al, Ind. J. Chem., 1969, 7, 1195-1197).

### Preparation (5b) methyl {2-[4-(2-methoxyphenyl)piperazine-1-yl]-4-phenylthiazol-5-yl}acetate

A solution of 4-(2-methoxyphenyl)piperazinecarbothioic acid amide (0.13 g) and methyl 3-bromo-4-oxo-4-phenylbutanoate (0.14 g) in ethanol was heated at reflux for three hours. The reaction mixture was concentrated under reduced pressure and the residue was dissolved in a saturated aqueous sodium hydrogen carbonate solution (10 mL) and extracted with ethyl acetate. The combined extracts were washed with saturated aqueous sodium chloride solution, dried over sodium sulphate, and concentrated under reduced pressure. The oily residue was purified by flash chromatography on silica gel eluting with a mixture of petroleum ether, ethyl acetate and dichloromethane (3:1:1 by volume) to give methyl {2-[4-(2-methoxyphenyl)piperazine-1-yl]-4-phenylthiazol-5-yl}acetate as a white foam, 0.18 g

### Preparation (5c) {2-[4-(2-methoxyphenyl)piperazine-1-yl]-4-phenylthiazol-5-yl}acetic acid

Methyl {2-[4-(2-methoxyphenyl)piperazine-1-yl]-4-phenylthiazol-5-yl}acetate (0.18 g) was added to a solution of sodium hydroxide (30 mg) in ethanol (3.0 mL) and water (3.0 mL), and the resulting mixture was stirred at room temperature for twenty hours. The mixture was concentrated to low bulk and the residue was treated with water (5.0 mL) followed by acidification to pH 5.5-6 by the addition of dilute hydrochloric acid. The resulting precipiate was extracted with ethyl acetate and the organic layer was washed with saturated aqueous sodium chloride solution, dried over sodium sulphate, and concentrated under reduced pressure. The crude product was purified by flash chromatography on silica gel, eluting with a mixture of dichloromethane and methanol (20:1 by volume) to give {2-[4-(2-methoxyphenyl)piperazine-1-yl]-4-phenylthiazol-5-yl}acetic acid as a white foam, 0.60 g.

¹H NMR (DMSO-d6; Method B): δ 3.05 (m, 4H), 3.50 (m, 4H), 3.65 (s, 2H), 3.75 (s, 3H), 6.80-6.95 (m, 4H), 7.25-7.30 (m, 1H), 7.35-7.40 (m, 2H), 7.50 (m, 2H).

MS: ESI (+ve): 410 (M+H)⁺, Retention time 10.5 min.

### Example 6

### [2-(4-benzylpiperazin-1-yl)-4-phenylthiazol-5-yl]acetic acid

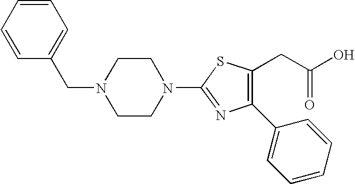

### Preparation (6a) 4-benzylpiperazinecarbothioic acid amide

4-Benzylpiperazinecarbothioic acid amide was prepared following a literature procedure (Nagarajan et al., Ind. J. Chem., 1969, 7, 1195-1197).

### Preparation (6b) methyl [2-(4-benzylpiperazin-1-yl)-4-phenylthiazol-5-yl]acetate hydrobromide

A mixture of 4-benzylpiperazinecarbothioic acid amide (0.17 g) and methyl 3-bromo-4-oxo-4-phenylbutanoate (0.20 g) in ethanol (10 mL) was heated at reflux for one hour, and then left to stand at room temperature overnight. The mixture was heated at reflux for a further two hours and then allowed to cool to room temperature. The resultant precipitate was collected by filtration, washed with anhydrous ethanol, and dried to give methyl [2-(4-benzylpiperazin-1-yl)-4-phenylthiazol-5-yl]acetate hydrobromide as a white powder, 0.18 g.

¹H NMR (CDCl₃; Method A): δ: 3.32-3.55 (m, 4H), 3.74 (s, 2H), 3.77 (s, 2H), 4.16-4.48 (m, 6H), 7.37-7.58 (m, BH), 7.60-7.74 (m, 2H).

### Preparation (6c) [2-(4-benzylpiperazin-1-yl)-4-phenylthiazol-5-yl]acetic acid

Methyl [2-(4-benzylpiperazin-1-yl)-4-phenylthiazol-5-yl]acetate hydrobromide (0.17 g) was treated with a solution of lithium hydroxide (0.03 g) in water (2.0 mL) and ethanol (4.0 mL) and the resulting mixture was stirred at 60° C. for two hours. The mixture was allowed to stand at room temperature overnight and then filtered. The filtrate was concentrated to low bulk and the pH of the residue was adjusted to 6 by the addition of dilute hydrochloric acid. The resulting precipitate was collected by filtration, washed with water, and dried to give [2-(4-benzylpiperazin-1-yl)-4-phenylthiazol-5-yl]acetic acid as a white powder, 0.12 g.

¹H NMR (DMSO-d6; Method B): δ 2.55-2.60 (m, 2H), 3.30-3.40 (m, 8H), 3.70 (s, 2H), 7.30-7.40 (m, 8H), 7.50 (m, 2H).

MS: ESI (+ve): 394 (M+H)⁺, Retention time 6.8 min.

### Example 7

### N-{2-[4-phenyl-2-(4-phenylpiperazin-1-yl)thiazol-5-yl]acetyl}methanesulphonamide

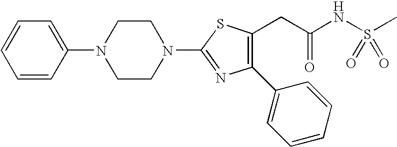

### Preparation (7a) N-{2-[4-phenyl-2-(4-phenylpiperazin-1-yl)thiazol-5-yl]acetyl}-methanesulphonamide

A mixture of [4-phenyl-2-(4-phenylpiperazin-1-yl)thiazole-5-yl]acetic acid (compound of Preparation (4b), 0.060 g), 1-(3-dimethylaminopropyl)-3-ethylcarbodiimide hydrochloride (0.038 g), methanesulphonamide (0.019 g), and 4-(dimethylamino)pyridine (0.024 g) in dichloromethane (5 mL) was stirred at room temperature for twenty four hours. The mixture was concentrated and the residue was purified by flash chromatography on silica gel, eluting with dichloromethane followed by ethyl acetate to give a yellow solid. Further purification by preparative reverse-phase HPLC using a gradient over 30 minutes of acetonitrile in water (10% to 90% of organic modifier) gave N-{2-[4-phenyl-2-(4-phenylpiperazin-1-yl)thiazol-5-yl]acetyl}methanesulphonamide trifluoroacetate salt as a white powder, 0.028 g.

¹H NMR (DMSO-d6; Method B): δ 3.25 (s, 3H), 3.30 (m, 4H), 3.55 (m, 4H), 3.85 (s, 2H), 6.85 (m, 1H), 7.00 (m, 2H), 7.25 (m, 2H), 7.35-7.40 (m, 1H), 7.45 (m, 2H), 7.55 (m, 2H), 12.00 (br s, 1H).

MS: ESI (+ve): 457 (M+H)⁺, Retention time 11.2 min.

### Example 8

### N-(3-{2-[4-(4-methoxyphenyl)piperazin-1-yl]-4-phenylthiazol-5-yl}propionyl)benzenesulphonamide

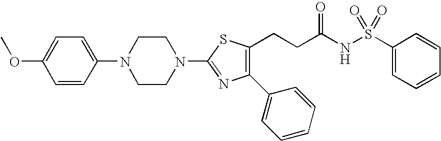

### Preparation (8a) N-(3-{2-[4-(4-methoxyphenyl)piperazin-1-yl]-4-phenylthiazol-5-yl}propionyl)benzenesulphonamide

N-(3-{2-[4-(4-methoxyphenyl)piperazin-1-yl]-4-phenylthiazol-5-yl}propionyl)benzenesulphonamide was prepared from benzenesulphonamide (0.015 g) and 3-{2-[4 (4-methoxyphenyl)piperazin-1-yl]-4-phenylthiazol-5-yl}propionic acid (compound of Preparation (3b), 0.033 g) using the method described in Preparation (7a). The crude product was purified by preparative reverse-phase HPLC using a gradient over 30 minutes of acetonitrile in water (10% to 90% of organic modifier) to give N-(3-{2-[4-(4-methoxyphenyl)piperazin-1-yl]-4-phenylthiazol-5-yl}propionyl)benzenesulphonamide trifluoroacetate salt as a pale yellow solid, 0.031 g.

¹H NMR (DMSO-d6; Method B): δ 2.55 (t, J=7.1 Hz, 2H), 2.90 (t, J=7.1 Hz, 2H), 3.20 (m, 4H), 3.45 (m, 4H), 3.70 (s, 3H), 6.85 (m, 2H), 7.00 (m, 2H), 7.30-7.35 (m, 1H), 7.40 (m, 2H), 7.45-7.50 (m, 2H), 7.60-7.65 (m, 2H), 7.70-7.75 (m, 1H), 7.90 (m, 2H), 12.20 (br s, 1H).

MS: ESI (+ve): 563 (M+H)⁺, Retention time 11.9 min.

### Example 9

### N-(2-{2-[4-(4-methoxyphenyl)piperazine-1-yl]-4-phenylthiazol-5-yl}acetyl)benzenesulphonamide

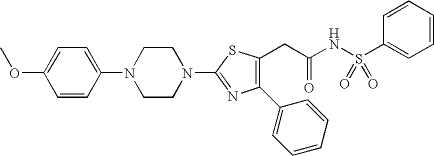

### Preparation (9a) N-(2-{2-[4-(4-methoxyphenyl)piperazine-1-yl]-4-phenylthiazol-5-yl}acetyl)benzenesulphonamide

N-(2-{2-[4-(4-methoxyphenyl)piperazine-1-yl]-4-phenylthiazol-5-yl}acetyl)benzenesulphonamide was prepared from benzenesulphonamide (0.019 g) and {2-[4-(4-methoxyphenyl)piperazine-1-yl]-4-phenylthiazol-5-yl}acetic acid (0.039 g) using the method described in Preparation (7a). The crude product was purified by preparative reverse-phase HPLC using a gradient over 30 minutes of acetonitrile in water (10% to 90% of organic modifier) to give N-2-{2-[4-(4-methoxyphenyl)piperazine-1-yl]-4-phenylthiazol-5-yl}acetyl)benzenesulphonamide trifluoroacetate salt as a pale pink solid, 0.033 g.

¹H NMR (DMSO-d6; Method B): δ 3.10 (m, 4H), 3.50 (m, 4H), 3.65 (s, 3H), 3.70 (s, 2H), 6.80 (m, 2H), 6.95 (m, 2H), 7.30 (m, 5H), 7.60 (m, 2H), 7.70 (m, 1H), 7.90 (m, 2H), 12.40 (br s, 1H).

MS: ESI (+ve): 549 (M+H)⁺, Retention time 11.7 min.

### Example 10

### N-{2-[4-phenyl-2-(4-phenylpiperazin-1-yl)thiazole-5-yl]acetyl}benzene sulphonamide

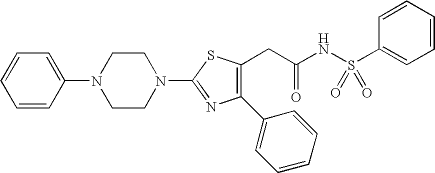

### Preparation (10a) N-{2-[4-phenyl-2-(4-phenylpiperazin-1-yl)thiazole-5-yl]acetyl}benzenesulphonamide

N-{2-[4-phenyl-2-(4-phenylpiperazin-1-yl)thiazole-5-yl]acetyl}benzenesulphonamide was prepared from benzenesulphonamide (0.031 g) and [4-phenyl-2-(4-phenylpiperazin-1-yl)thiazole-5-yl]acetic acid (compound of Preparation (4b), 0.060 g) using the method described in Preparation (7a). The crude product was purified by preparative reverse-phase HPLC using a gradient over 30 minutes of acetonitrile in water (10% to 90% of organic modifier) to give N-{2-[4-phenyl-2-(4-phenylpiperazin-1-yl)thiazole-5-yl]acetyl}benzenesulphonamide trifluoroacetate salt as a pale pink solid, 0.027 g.

¹H NMR (DMSO-d6; Method B): δ 3.20 (m, 4H), 3.50 (m, 4H), 3.70 (s, 2H), 6.75 (m, 1H), 6.95 (m, 2H), 7.20 (m, 2H), 7.30 (m, 5H), 7.60 (m, 2H), 7.70 (m, 1H), 7.90 (m, 2H), 12.35 (br s, 1H).

MS: ESI (+ve): 519 (M+H)⁺, Retention time 12.6 min.

### Example 11

### 4-phenyl-2-(4-phenylpiperidin-1-yl)thiazole-5-carboxylic acid

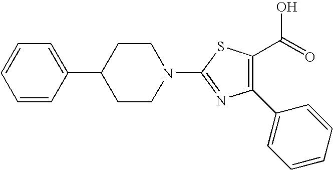

### Preparation (11a) Ethyl 2-chloro-4-phenylthiazol-5-carboxylate

tert-Butyl nitrite (14.4 g) was added to stirred suspension of copper (II) chloride (16 g) in acetonitrile (450 mL). The mixture was warmed to 65° C. and a suspension of ethyl 2-amino-4-phenylthiazol-5-carboxylate (25 g) in acetonitrile (150 mL) was added slowly over one hour whilst maintaining the temperature at 65° C. On completion of the addition, the mixture was stirred at 65° C. for a further thirty minutes. After cooling to room temperature the mixture was poured onto 1 M aqueous hydrochloric acid and extracted with ethyl acetate. The combined extracts were washed with saturated aqueous sodium chloride solution, dried over magnesium sulphate, and concentrated to give ethyl 2-chloro-4-phenylthiazol-5-carboxylate as an orange solid, 22 g.

### Preparation (11b) 4-phenyl-2-(4-phenylpiperidin-1-yl)thiazole-5-carboxylic acid

A mixture of ethyl 2-chloro-4-phenylthiazol-5-carboxylate (0.25 g), triethylamine (0.66 mL), and 4-phenylpiperidine (0.15 g) in N,N-dimethylformamide (5 mL) was stirred at room temperature overnight. The mixture was concentrated and the residue purified by flash chromatography on silica gel, eluting with a gradient mixture of ethyl acetate and hexane (3:7 to 1:0 by volume) to give a white solid. This material was dissolved in methanol (20 mL) and treated with a solution of sodium hydroxide (0.75 g) in water (10 mL) and the mixture stirred at 80° C. for four hours. The solvent was removed under reduced pressure and the residue was dissolved in water. The pH of this solution was adjusted to about 5 by the addition of acetic acid and the resultant precipitate was collected by filtration, washed with water and dried to give 4-phenyl-2-(4-phenylpiperidin-1-yl)thiazole-5-carboxylic acid as a white solid, 0.28 g.

¹H NMR (DMSO-d6; Method B): δ 1.65-1.75 (m, 2H), 1.85 (m, 2H), 2.75-2.80 (m, 1H), 3.20 (m, 2H), 4.05 (m, 2H), 7.15 (m, 1H), 7.20-7.30 (m, 4H), 7.30-7.35 (m, 3H), 7.60-7.65 (m, 2H), 12.40 (br s, 1H).

MS: ESI (+ve): 365 (M+H)⁺, Retention time 12.6 min.

### Example 12

### 2-[4-(2-methoxyphenyl)piperazine-1-yl]-4-phenylthiazole-5-carboxylic acid

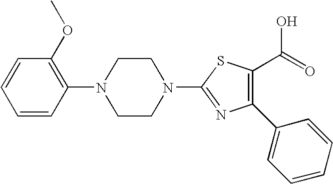

### Preparation (12a) Ethyl 2-[4-(2-methoxyphenyl)piperazine-1-yl]-4-phenylthiazole-5-carboxylate

A mixture of ethyl 2-chloro-4-phenylthiazol-5-carboxylate (5.0 g), triethylamine (9.9 mL), and 2-methoxyphenylpiperazine hydrochloride (5.3 g) in N,N-dimethylformamide (50 mL) was stirred at room temperature for two days. The mixture was concentrated and the residue purified by flash chromatography on silica gel, eluting with dichloromethane to give ethyl 2-[4-(2-methoxyphenyl)piperazine-1-yl]-4-phenylthiazole-5-carboxylate as a yellow oil, 2.9 g.

¹H NMR (DMSO-d6; Method B): δ 1.10 (t, J=7.1 Hz, 3H), 3.05 (m, 4H), 3.65 (m, 4H), 3.75 (s, 3H), 4.10 (q, J=7.1 Hz, 2H), 6.80-7.00 (m, 4H), 7.35 (m, 3H), 7.65 (m, 2H).

MS: ESI (+ve): 424 (M+H)⁺, Retention time 14.6 min.

### Preparation (12b) 2-[4-(2-methoxyphenyl)piperazine-1-yl]-4-phenylthiazole-5-carboxylic acid

A mixture of ethyl 2-[4-(2-methoxyphenyl)piperazine-1-yl]-4-phenylthiazole-5-carboxylate (0.25 g) was added to a solution of sodium hydroxide (0.24 g) in water (5.0 mL) and methanol (10 mL) and the resulting mixture stirred at room temperature for two days. The mixture was concentrated to low bulk and the residue dissolved in water. The pH of the solution was adjusted to about 5 by the addition of acetic acid and the resultant precipitate was collected by filtration, washed with water and dried to give 2-[4-(2-methoxyphenyl)piperazine-1-yl]-4-phenylthiazole-5-carboxylic acid, 0.23 g.

¹H NMR (DMSO-d6; Method B): δ 3.00 (m, 4H), 3.60 (m, 4H), 3.75 (s, 3H), 6.80-7.00 (m, 4H), 7.30-7.35 (m, 3H), 7.65-7.70 (m, 2H).

MS: ESI (+ve): 396 (M+H)⁺, Retention time 11.4 min.

### Example 13

### 1-(4-methoxyphenyl)-4-[4-phenyl-5-(1H-tetrazol-5-yl)thiazol-2-yl]piperazine

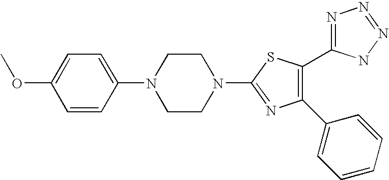

### Preparation (13a) 2-[4-(4-methoxyphenyl)piperazin-1-yl]-4-phenylthiazole-5-carboxylic acid amide

A mixture of 2-[4-(4-methoxyphenyl)piperazin-1-yl]-4-phenylthiazol-5-carboxylic acid (compound of Preparation (1b), 3.0 g), tetrahydrofuran (60 mL), thionyl chloride (0.83 mL) and N,N-dimethylformamide (3 drops) were stirred at room temperature for one hour. The mixture was poured into 28% aqueous ammonium hydroxide solution and stirred at room temperature for one hour. The precipitate was collected by filtration to give 2-[4-(4-methoxyphenyl)piperazin-1-yl]-4-phenylthiazole-5-carboxylic acid amide as a white powder, 1.25 g.

¹H NMR (DMSO-d6; Method B): δ 3.15 (m, 4H), 3.60 (m, 4H), 3.70 (s, 3H), 6.85 (m, 2H), 6.95 (m, 2H), 7.40-7.45 (m, 3H), 7.60-7.65 (m, 2H).

MS: ESI (+ve): 395 (M+H)⁺, Retention time 9.9 min.

### Preparation (13b) 2-[4-(4-methoxyphenyl)piperazin-1-yl]-4-phenylthiazole-5-carbonitrile

A mixture of 2-[4-(4-methoxyphenyl)piperazin-1-yl]-4-phenylthiazole-5-carboxylic acid amide (compound from Preparation (13a), 1.0 g), N,N-dimethylformamide (10 mL) and phosphorus oxychloride (0.58 mL) were heated at 100° C. for four hours. After cooling to room temperature, the mixture was poured into a mixture of ice and water. The pH of the solution was adjusted to 7 with a saturated aqueous sodium hydrogencarbonate solution, and extracted with ethyl acetate. The organic layer was washed with water, and saturated aqueous sodium chloride solution, dried over magnesium sulfate and concentrated under reduced pressure. The residue was purified by column chromatography on silica gel, eluting with a mixture of ethyl acetate and dichloromethane (20:1 by volume), to give 2-[4-(4-methoxyphenyl)piperazin-1-yl]-4-phenylthiazole-5-carbonitrile as a pale yellow solid, 0.55 g.

¹H NMR (CDCl₃; Method B): δ: 3.20 (m, 4H), 3.80 (m, 7H), 6.85 (m, 2H), 6.95 (m, 2H), 7.45-7.50 (m, 3H), 8.05-8.10 (m, 2H).

### Preparation (13c) 1-(4-methoxyphenyl)-4-[4-phenyl-5-(1H-tetrazol-5-yl)thiazol-2-yl]piperazine

A mixture of 2-[4-(4-methoxyphenyl)piperazin-1-yl]-4-phenylthiazole-5-carbonitrile (0.15 g), sodium azide (0.13 g), ammonium chloride (0.11 g) and N,N-dimethylformamide (5.0 mL) were heated at 100° C. for nine hours in a microwave reactor. The mixture was concentrated and the residue purified by preparative reverse-phase HPLC using a gradient over 30 minutes of acetonitrile in water (30% to 70% of organic modifier) to give 1-(4-methoxy-phenyl)-4-[4-phenyl-5-(1H-tetrazol-5-yl)-thiazol-2-yl]-piperazine (5 mg) as a white solid.

¹H NMR (DMSO-d6; Method B); δ 3.10 (m, 4H), 3.60-3.65 (m, 7H), 6.80 (m, 2H), 6.95 (m, 2H), 7.35 (m, 3H), 7.50 (m, 2H).

MS: ESI (−ve): 418 (M−H)⁻, Retention time 10.7 min.

1. A compound for therapeutic use of structural formula [1]: 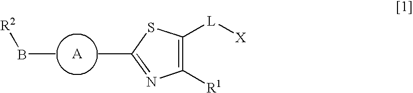
in which:
A represents a fully saturated or partially unsaturated monocyclic 5-7 membered ring containing one or two nitrogen atoms;
B represents a direct bond, an optionally substituted methylene group, an optionally substituted nitrogen atom, oxygen, or S(O)ₙ, where n=0, 1, or 2;
L represents a direct bond, or an optionally substituted alkylene or alkenylene group;
R¹ represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
R² represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
X represents a carboxylic acid, tetrazole, 3-hydroxyisoxazole, hydroxamic acid, phosphinate, phosphonate, phosphonamide, sulfonic acid or a group of formula C(═O)NHSO₂Y or SO₂NHC(═O)Y; and
Y represents an optionally substituted aryl or heteroaryl group or an optionally substituted alkyl or cycloalkyl group;
or a N-oxide, pharmaceutically acceptable salt, solvate or prodrug thereof.

2. The compound as claimed in claim 1, wherein A is a radical of formula (2), (3) or (5):

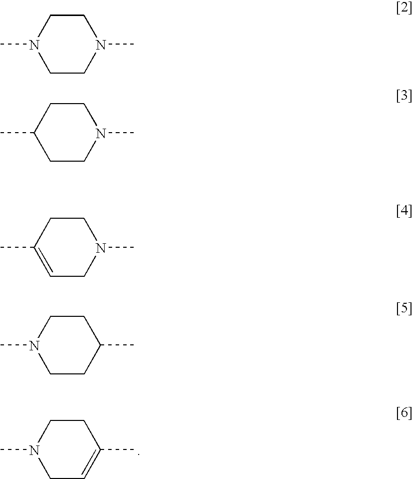

3. The compound as claimed in claim 1, wherein L is a bond, or a divalent radical selected from —CH₂—, —CH₂CH₂—, —CH₂CH₂CH₂—, —CH═CH—, —CH═CHCH₂— and —CH₂CH═CH—.

4. The compound as claimed in claim 1, wherein Z is selected from —COOH and 5-tetrazolyl.

5. The compound as claimed in claim 1, wherein B is a bond or —CH₂—.

6. The compound as claimed in claim 1, wherein R¹ is optionally substituted phenyl.

7. The compound as claimed in claim 1, wherein R² is optionally substituted phenyl.

8. The compound as claimed in claim 7, wherein any optional substituents in R¹ and R² are selected from C₁-C₃alkoxy, halo, cyano, C₁-C₃-alkyl, C₁-C₃-acylamino, and mono- or di-C₁-C₃-alkylamino.

9. (canceled)

10. A pharmaceutical composition comprising a compound of structural formula [1]: 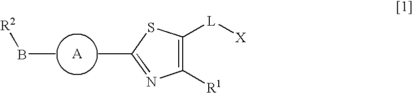
in which:
A represents a fully saturated or partially unsaturated monocyclic 5-7 membered ring containing one or two nitrogen atoms;
B represents a direct bond, an optionally substituted methylene group, an optionally substituted nitrogen atom, oxygen, or S(O)ₙ, where n=0, 1, or 2;
L represents a direct bond, or an optionally substituted alkylene or alkenylene group;
R¹ represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
R² represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
X represents a carboxylic acid, tetrazole, 3-hydroxyisoxazole, hydroxamic acid, phosphinate, phosphonate, phosphonamide, sulfonic acid or a group of formula C(═O)NHSO₂Y or SO₂NHC(═O)Y; and
Y represents an optionally substituted aryl or heteroaryl group or an optionally substituted alkyl or cycloalkyl group;
or a N-oxide, pharmaceutically acceptable salt, solvate or prodrug thereof, and a carrier.

11-13. (canceled)

14. A method of preventing, treating or ameliorating a disease mediated by the PGD₂ receptor in a subject in need thereof comprising the administration of a therapeutically effective amount of a compound of structural formula [1]: 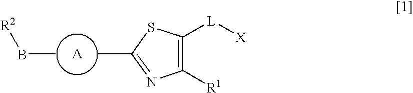
in which:
A represents a fully saturated or partially unsaturated monocyclic 5-7 membered ring containing one or two nitrogen atoms;
B represents a direct bond, an optionally substituted methylene group, an optionally substituted nitrogen atom, oxygen, or S(O)ₙ, where n=0, 1, or 2;
L represents a direct bond, or an optionally substituted alkylene or alkenylene group;
R¹ represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
R² represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
X represents a carboxylic acid, tetrazole, 3-hydroxyisoxazole, hydroxamic acid, phosphinate, phosphonate, phosphonamide, sulfonic acid or a group of formula C(═O)NHSO₂Y or SO₂NHC(═O)Y; and
Y represents an optionally substituted aryl or heteroaryl group or an optionally substituted alkyl or cycloalkyl group;
or a N-oxide, pharmaceutically acceptable salt, solvate or prodrug thereof.

15. The method of claim 14 wherein the disease is selected from asthma, allergic rhinitis, allergic conjunctivitis and atopic dermatitis.

16. The method of claim 14, wherein the disease is selected from Crohn's disease, ulcerative colitis, sleep disorders and cancer.

17. A compound of structural formula [1]: 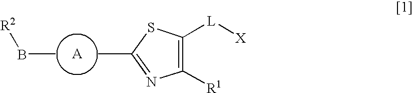
in which:
A represents a fully saturated or partially unsaturated monocyclic 5-7 membered ring containing one or two nitrogen atoms;
B represents a direct bond, an optionally substituted methylene group, an optionally substituted nitrogen atom, oxygen, or S(O)ₙ, where n=0, 1, or 2;
L represents a direct bond, or an optionally substituted alkylene or alkenylene group;
R¹ represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
R² represents an optionally substituted aryl or heteroaryl group, or an optionally substituted aryl-fused-heterocycloalkyl, heteroaryl-fused-cycloalkyl, heteroaryl-fused-heterocycloalkyl or aryl-fused-cycloalkyl group;
X represents a carboxylic acid, tetrazole, 3-hydroxyisoxazole, hydroxamic acid, phosphinate, phosphonate, phosphonamide, sulfonic acid or a group of formula C(═O)NHSO₂Y or SO₂NHC(═O)Y; and
Y represents an optionally substituted aryl or heteroaryl group or an optionally substituted alkyl or cycloalkyl group;
or a N-oxide, pharmaceutically acceptable salt, solvate or prodrug thereof, excluding {2-[4-(4-methoxyphenyl)piperazin-1-yl]-4-phenylthiazol-5-yl}acetic acid.
